(19) United States
(12) Patent Application Publication
(10) Pub. No.: US 2026/0337640 A1
(43) Pub. Date: Oct. 29, 2026

(54) MUTATION-MEDIATED DETECTION OF CANCERS USING SYNTHETIC BIOMARKER PRODUCTION

(71) Applicant: William Marsh Rice University, Houston, TX (US)

(72) Inventors: Kevin J. McHUGH, Houston, TX (US); Shengyue PIAO, Houston, TX (US); Tyler P. GRAF, Houston, TX (US)

(73) Assignee: William Marsh Rice University, Houston, TX (US)

(21) Appl. No.: 19/655,616

(22) Filed: Apr. 22, 2026

Related U.S. Application Data
(60) Provisional application No. 63/793,926, filed on Apr. 24, 2025.

(51) Int. Cl. C12N 15/90; C12N 9/22; C12N 15/11; G01N 33/57525

(52) U.S. Cl. CPC C12N 15/907; C12N 9/226; C12N 15/11; G01N 33/57525; C12N 2310/20; G01N 2333/43504; G01N 2333/90241

(57) ABSTRACT

The present disclosure is directed to compositions and methods for the labeling of cancer cells with detectable markers. Using genetic editing tools, cancer cells harboring specific mutations can be modified, based on the presence of such mutations, with signal-producing or otherwise detectable proteins that will only, or nearly only, be expressed by cells having the mutations. This will permit early detection and diagnosis, imaging and monitoring of cancer progression/treatment.

### PRIORITY CLAIM

This application claims benefit of priority to U.S. Provisional Application Ser. No. 63/793,926, filed Apr. 24, 2025, the entire contents of which are hereby incorporated by reference.

### BACKGROUND

### 1. Field of the Disclosure

The present disclosure relates generally to the fields of molecular biology, genetics, oncology, medicine and diagnostics. More particularly, the disclosure relates to the targeting of mutated cancer cells with gene editing approaches to induce the expression of markers for detection, diagnosis and treatment.

### 2. Background

Cancer kills an estimated 10 million people each year and costs $1.16 trillion annually to treat (GDB 2016 Causes of Death Collaborators, 2017; de Souza et al., 2016). One of the most critical factors determining patient outcomes is the stage at which the cancer is detected (Crosby et al., 2022). Although there are some exceptions, the prognosis is generally far more favorable when diagnosed while the cancer remains localized due to the lack of effective treatments for most regional and distal cancers (Crosby et al., 2022; Kaiser, 2018). For some types of cancer, distal diagnosis is associated with a >75% reduction in five-year survival. Therefore, there is a clear clinical need for accelerated cancer diagnostics that will dramatically improve survival. Even if a diagnostic were only applied to the ten most common cancers, broad localized diagnosis could prevent nearly 225,000 cancer deaths (55%) each year in the U.S.

Unfortunately, cancer is a highly heterogeneous disease with variability across different types of cancer, between individuals, and even within an individual's tumor, making the development of diagnostics that are both broadly applicable and highly specific exceptionally difficult (Cleeland et al., 2012). At present, the most widely used cancer screening methods include colonoscopies for colorectal cancer, mammograms for breast cancer, and pap smears for cervical cancer, which are far from ideal since they only detect a small subset of cancers when they are sufficiently advanced to generate a visually obvious tumor, are expensive, and are often invasive. Therefore, there is a clear need to diagnose cancers at an earlier stage when they remain at a nascent stage.

### SUMMARY

Thus, in accordance with the present disclosure, there is provided a method of labeling a cancer cell comprising subjecting the cancer cell to gene editing that introduces a genetic element encoding a detectable marker peptide or protein when one or more cancer-specific mutations are present. The gene editing may comprise Cre-Lox recombination, Prime Editing, Base Editing, TALEN editing, zinc finger nuclease editing, CRISPR/Cas editing, e.g., PASSIGE™, or an RNA-editing technique, e.g., Cas13a. The detectable marker peptide or protein may be a surface-bound or extracellular matrix-bound target, such as bindin, and detecting comprises contacting said cancer cell with an target binding agent, such as antibody, an aptamer, or bioconjugate pair (e.g., SpyTag/SpyCatcher) optionally wherein said target binding agent is labeled with a detectable moiety, or wherein the detectable marker peptide or protein is an intracellular protein. The detectable marker peptide or protein may be an enzyme, such as luciferase. The detectable peptide or protein may be linked to a reverse-transcytosis domain, e.g., FcRn, Dectin-1, Siglec-5, or TOSO/FAIM3 to increase the sensitivity of detection by increasing the half-life of the detectable protein. The method may further comprise detecting a signal produced directly or indirectly from the presence of said detectable peptide or protein marker.

The labeling may be performed in vitro on a cancer cell obtained from a subject, optionally wherein detecting comprises an ELISA, lateral flow assay, a bioluminescence assay, chemical assay (e.g., liquid chromatography-mass spectrometry), or absorbance-based assay, or may be performed in vivo in a subject, such as intratumorally, interperitoneally, or intravenously. The cancer cell may be contacted with a candidate substance and cultured, wherein a lower signal, as compared to an untreated cancer cell having the same genetic abnormality and cultured under the same conditions, indicates that said candidate substance has anti-cancer activity. Detecting comprises obtaining a body fluid from the subject, such as blood, saliva, sputum, urine, semen or other bodily secretion, optionally wherein detecting comprises an ELISA, lateral flow assay, a bioluminescence assay, chemical assay (e.g., liquid chromatography-mass spectrometry), or absorbance-based assay. Detecting may also comprise obtaining an image from the subject. The target binding agent may be labeled with a contrast agent, a radioisotope, a molecular imaging agent, or a nanoparticle. The imaging may comprise detecting an enzymatic signal, a bioluminescent signal, a chemical signal, or an absorbance-based signal.

The coding region for said detectable marker peptide or protein may be operatively linked to an inducible promoter, a constitutively active promoter, or does not have a promoter operatively linked, or involve trigger Cre-Lox recombination events. The gene editing may insert more than one genetic element encoding more than one detectable marker peptide or protein at more than one mutation site, such as wherein said multiple detectable marker peptides or proteins each produce distinguishable signals.

A cancer cell from said subject diagnosed with cancer may have been sequenced to determine the presence of a mutation. The method may further comprise making a diagnosis, treatment decision, or treatment based on a location and/or extent of said signal. The method may further comprise performing detecting said signal a second time determining a change in a previously obtained signal following treatment. The subject may have been previously diagnosed and treated for cancer, such as wherein said subject has been in remission, or the subject may not have been diagnosed with cancer and has no known cancer risk factors. The subject may not have been diagnosed with cancer and has one or more known cancer risk factors.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. Generalized biomarker gene insertion strategy. Schematics of (FIG. 1A) mutation-targeted CRISPR insertion of donor DNA into cancer cells, (FIG. 1B) secNLuc gene insertion and protein secretion in melanoma cells harboring the target mutation, and (FIG. 1C) bindin gene insertion and protein expression on the melanoma cell surface, enabling detection and localization via magnetic resonance imaging (MRI).

FIG. 2. VQR PAM sites emerged due to the KRAS G12D and BRAF V600E mutations. Genome-editing formulations are customized to target and cut sequences present in cancer cells due to mutation while minimizing their propensity to cut the corresponding wild-type sequence and other off-target sequences.

FIG. 3. Mechanism of Utilizing CRISPR/homology Directed Repair to Knock-in Gaussia luciferase gene. Following on-target cutting, a donor gene encoding a biomarker is inserted into the gene.

FIG. 4. Design and Selection of gRNAS of KRAS G12v mutation in NCI-H727 cells. Suitable gRNAs are produced and screened for cutting efficiency and specificity.

FIG. 5. Luminescence signal from cell colonies. Graph showing the distribution of luciferase expression across clonal cell populations.

FIG. 6. Sanger sequencing confirms the insertion into genomic DNA at KRAS G12 position. Sanger sequencing of the insertion site from clones found to express Gaussia luciferase shows that 21/29 colonies had the correctly inserted sequence.

FIG. 7. Relative expression analysis of KRAS G12-G-Luc and total G-Luc via qPCR. Use of two pairs of primers: one pair is out-in, and the other is in-in. The term ‘out-in’ refers to expression of G-Luc at the Kras gene, while ‘in-in’ refers to the total expression of G-Luc in cells. Ten S10 colonies were picked for subsequent study since the ratio is around 1. Samples S2, S7, S14, S18, and S21 should be excluded as their standard deviation (SD)>0.5. Overall, a comparison of gene amplification levels suggests the unique on-target insertion of G-Luc in several clonal cell populations.

FIG. 8. Demonstration of G-Luc-induced bioluminescence by edited cells in vitro. Edited cells containing the gene encoding G-Luc exhibit substantial bioluminescence when treated with a luciferase substrate compared to unedited cells, which increases over time due to luciferase accumulation and cell expansion.

FIG. 9. Demonstration of the ability to detect G-Luc biomarkers produced by edited cells in vivo. Serum isolated from blood shows high levels of luciferase present in mice harboring edited tumor cells. Circle indicates tumor inoculation site (10⁶ cell seeding); square is control.

FIG. 10. Increasing the half-life of G-Luc in vivo using reverse transcytosis domain. The reverse transcytosis domain (RTD) is a protein or peptide motif that can interact with Fc receptor (FcRn) or other similar receptors involved in transcytosis. The ‘reverse’ aspect can refer to recycling the protein back to circulation instead of being degraded, increasing its biological half-life. FcRn transports IgG antibodies from the blood into tissues and also protects them from lysosomal degradation. The half-life (t_{1/2}) of Gluc-mIgG1Fc in the blood is approximately 4 days.

FIG. 11. Luminescence increased >20-fold in vivo. The G-Luc-IgG1Fc group exhibited luminescence that was over 20-fold higher than those of the G-Luc group at approximately 30 days, indicating potentially higher sensitivity.

FIG. 12. High-Specificity Early Cancer Detection via eePASSIGE. (Left) By utilizing the eePASSIGE gene editing system, an off-target (unspecific) insertion rate of <0.08% was achieved, thereby enhancing specificity. (Right) Efficient insertion of eGFP into the KRAS G12V locus in NCI-H727 cells following puromycin selection.

FIG. 13. Urease insertion into cells can generate contrast via hyperpolarized MRI for cancer detection. Utilizing the eePAssige gene-editing platform, we achieved precise insertion of the Urease gene at the KRAS G12V locus in NCI-H727 cells, facilitating sensitive tumor detection through hyperpolarized MRI.

### DESCRIPTION OF ILLUSTRATIVE EMBODIMENTS

As discussed above, cancer is the second leading cause of death worldwide, killing more than 10 million people annually. Treating cancer is especially difficult due to several factors, including the high variability between cancers, the high similarity between cancer cells and normal cells, and the need to eradicate every cancer cell to achieve a cure. As a result, treating cancer at an early stage when it remains localized and treatment is more effective leads to much lower mortality than when cancer has already metastasized. Current cancer screening techniques have relatively low sensitivity before cancer can be identified, and often occurs after the tumor has spread. Additionally, these methods are often invasive, expensive, identify only a small subset of cancers, and require ample skill to perform.

The inventors sought to develop a highly specific, broadly applicable, and widely accessible diagnostic for all types of cancer. They proposed using gene editing tools to insert genes and subsequently detect protein biomarkers only when specific cancer mutations are present, thus turning undetectable cancers into detectable ones. This strategy can be used to detect cancer based on the presence of cancerous mutations that have been tagged with one or more detectable protein biomarkers.

These and other aspects of the disclosure are described in detail below.

### I. Gene Editing Tools

The present disclosure relies in part on genome editing, or genome engineering, or gene editing, a type of genetic engineering in which DNA or RNA is inserted, deleted, modified or replaced in a living organism. Unlike early genetic engineering techniques that randomly insert genetic material into a nucleic acid target at site-specific locations. The basic mechanism involved in genetic manipulations through programmable nucleases is the recognition of targets. Here, targeted genome editing techniques are used to selectively integrate genes of interest into cells in a site-specific manner. Briefly, genome-editing formulations customized to the target mutation(s) are administered and, upon identifying their target sequence, cut nucleic acid, providing an opportunity to insert a gene of interest. This technique leverages the appearance of a unique target nucleotide sequence or sequences that arise due to substitution, insertion, deletion, or fusion events to enable editing in cancer cells, which harbor the mutation, without editing normal cells. In this manner, it selectively inserts the gene. Although any sequence could potentially be targeted, including non-synonymous mutations, mutations in oncogenes and tumor suppressor genes are likely among the best targets because they are the most likely to be conserved across cells within a patient's cancer. Consideration will also be given to the homology between the target sequence and sequences elsewhere in the genome.

There are a number of examples of gene editing tools that can be employed in accordance with the present disclosure. As of 2015, four families of engineered nucleases were used: meganucleases, zinc finger nucleases (ZFNs), transcription activator-like effector-based nucleases (TALEN), the clustered regularly interspaced short palindromic repeats (CRISPR/Cas9) system, as well as the Prime Editing and Base editing variations of CRISPR. Nine genome editors were available as of 2017.

An alternative to DNA editing approaches is RNA editing, which uses a molecular process through which some cells can make discrete changes to specific nucleotide sequences within an RNA molecule after it has been generated by RNA polymerase. It occurs in all living organisms and is one of the most evolutionarily conserved properties of RNAs. RNA editing may include the insertion, deletion, and base substitution of nucleotides within the RNA molecule. RNA editing is relatively rare, with common forms of RNA processing (e.g. splicing, 5′-capping, and 3′-polyadenylation) not usually considered as editing. It can affect the activity, localization as well as stability of RNAs, and has been linked with human diseases.

Cre-Lox Recombination. Cre-Lox recombination is a site-specific recombinase technology, used to carry out deletions, insertions, translocations and inversions at specific sites in the DNA of cells. It allows the DNA modification to be targeted to a specific cell type or be triggered by a specific external stimulus. It is implemented both in eukaryotic and prokaryotic systems. The Cre-lox recombination system has been particularly useful to help neuroscientists to study the brain in which complex cell types and neural circuits come together to generate cognition and behaviors. NIH Blueprint for Neuroscience Research has created several hundreds of Cre driver mouse lines which are currently used by the worldwide neuroscience community. The Cre-Lox system is very similar in action and in usage to the FLP-FRT recombination system.

An important application of the Cre-lox system is excision of selectable markers in gene replacement. Commonly used gene replacement strategies introduce selectable markers into the genome to facilitate selection of genetic mutations that may cause growth retardation. However, marker expression can have polar effects on the expression of upstream and downstream genes. Removal of selectable markers from the genome by Cre-lox recombination is an elegant and efficient way to circumvent this problem and is therefore widely used in plants, mouse cell lines, yeast, etc.

The system consists of a single enzyme, Cre recombinase, that recombines a pair of short target sequences called the Lox sequences. This system can be implemented without inserting any extra supporting proteins or sequences. The Cre enzyme and the original Lox site called the LoxP sequence are derived from bacteriophage P1.

Placing Lox sequences appropriately allows genes to be activated, repressed, or exchanged for other genes. At a DNA level many types of manipulations can be carried out. The activity of the Cre enzyme can be controlled so that it is expressed in a particular cell type or triggered by an external stimulus like a chemical signal or a heat shock. These targeted DNA changes are useful in cell lineage tracing and when mutants are lethal if expressed globally.

Cre-Lox recombination involves the targeting of a specific sequence of DNA and splicing it with the help of an enzyme called Cre recombinase. Cre-Lox recombination is commonly used to circumvent embryonic lethality caused by systemic inactivation of many genes. As of February 2019, Cre-Lox recombination is a powerful tool and is used in transgenic animal modeling to link genotypes to phenotypes.

The Cre-lox system is used as a genetic tool to control site specific recombination events in genomic DNA. This system has allowed researchers to manipulate a variety of genetically modified organisms to control gene expression, delete undesired DNA sequences and modify chromosome architecture.

The Cre protein is a site-specific DNA recombinase that can catalyse the recombination of DNA between specific sites in a DNA molecule. These sites, known as loxP sequences, contain specific binding sites for Cre that surround a directional core sequence where recombination can occur.

When cells that have loxP sites in their genome express Cre, a recombination event can occur between the loxP sites. Cre recombinase proteins bind to the first and last 13 bp regions of a lox site forming a dimer. This dimer then binds to a dimer on another lox site to form a tetramer. Lox sites are directional and the two sites joined by the tetramer are parallel in orientation. The double stranded DNA is cut at both loxP sites by the Cre protein. The strands are then rejoined with DNA ligase in a quick and efficient process. The result of recombination depends on the orientation of the loxP sites. For two lox sites on the same chromosome arm, inverted loxP sites will cause an inversion of the intervening DNA, while a direct repeat of loxP sites will cause a deletion event. If loxP sites are on different chromosomes it is possible for translocation events to be catalysed by Cre induced recombination. Two plasmids can be joined using the variant lox sites 71 and 66.

CRISPR Systems. Gene editing is a technology that allows for the modification of target genes within living cells. Recently, harnessing the bacterial immune system of CRISPR to perform on-demand gene editing revolutionized the way scientists approach genomic editing. The Cas9 protein of the CRISPR system, which is an RNA guided DNA endonuclease, can be engineered to target new sites with relative ease by altering its guide RNA sequence as well as engineered Cas9 variants or Cas9-family nucleases from other species. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a base editing enzyme or a reverse transcriptase.

The CRISPR enzyme can be Cas 9 (e.g., from S. pyogenes or S. pneumonia or S. aureus or S. auricularis or S. lugdunensis or an engineered enzyme with similar functionality). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas 9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce Non-Homologous End Joining or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, nucleic acid binding activity, base editing activity, or reverse transcription activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in U.S. Patent Publication 20110059502, incorporated herein by reference.

Base Editors. The engineered CRISPR technologies of base editing and prime editing have expanded the toolbox of gene editing strategies to potentially correct genetic mutations by enabling precise edits at individual nucleotides. In base editing, Cas9 nickase (nCas9) is fused to a deaminase protein, allowing precise single-base pair conversions without DSBs within a defined editing window in relation to the protospacer adjacent motif (PAM) site of a sgRNA. There are two major classes of DNA base editors: cytosine base editors (CBEs), which convert a C:G base pair into a T:A base pair, and adenine base editors (ABEs), which convert an A:T base pair into a G:C base pair. In instances where the programmable DNA-binding domain is a CRISPR/Cas nuclease, targeted adenines lie within an “editing window” in the single-stranded (ss) DNA bubble (R-loop) induced by the CRISPR-Cas RNA-protein complex. The most commonly used ABEs comprise an adenosine deaminase heterodimer consisting of E. coli TadA (wild type) fused to an engineered E. coli TadA variant (e.g. ABEmax) or a single engineered E. coli TadA variant (e.g. ABE8e, ABE8eV106W, or ABE8.20-m) as well as a nickase Cas9 and nuclear localization sequences (NLS). ABEs have been used successfully for installation of A-to-G substitutions in multiple cell types and organisms and could potentially reverse a large number of mutations known to be associated with human disease. Examples of ABEs include those described in U.S. Patent Publication US20200308571, PCT Publication WO2020214842, and PCT Publication WO2021025750, which are each incorporated herein by reference in their entirety. Reference is made to International Publication No. WO 2018/027078, published Aug. 2, 2018; International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; and U.S. Patent Publication No. 2017/0121693, published May 4, 2017, which issued as U.S. Pat. No. 10,167,457 on Jan. 1, 2019. One of the potential concerns reported for base editors is off-target editing. The present off-target analysis did not detect any significant off-target edits in the tested sites. Base editors, such as ABEmax, can edit all available base pairs within a defined activity window.

Prime Editors. Prime editing is a versatile and precise genome editing method that directly writes new genetic information into a specified DNA site using a CRISPR system working in association with a polymerase (i.e., in the form of a fusion protein or otherwise provided in trans with the CRISPR system), wherein the prime editing system is programmed with a prime editing (pe) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). As such, prime editors allow for prime editing on a target nucleotide sequence in the presence of a pegRNA (or “extended guide RNA”). The pegRNA consists of (from 5′ to 3′) a sgRNA that anneals to a target site, a scaffold for the nCas9, a reverse transcription template (RT template) containing the desired edit, and a primer binding site (PBS) that binds to the non-target strand. The RT template can be programmed to introduce any type of edit, including all possible base transitions and transversions, and insertions and deletions of nucleotides of any length. The prime editing system is further enhanced by including an additional nicking sgRNA that increases editing efficiency by favoring DNA repair to replace the non-edited strand. The term “prime editor” refers to fusion constructs comprising a Cas9 nickase and a reverse transcriptase. The term “prime editor” may refer to the fusion protein or to the fusion protein complexed with a pegRNA, and/or further complexed with a second-strand nicking sgRNA. In some embodiments, the prime editor may also refer to the complex comprising a fusion protein (reverse transcriptase fused to a Cas9), a pegRNA, and a regular guide RNA capable of directing the second-site nicking step of the non-edited strand as described herein. In other embodiments, the reverse transcriptase component of the “prime editor” may be provided in trans. Further examples of prime editors and their use are provided in PCT Publn. WO2020191249, which is incorporated by reference herein in its entirety.

While INDEL profiles from CRISPR-induced DSBs may have some sequence-dependent predictability in insertion and deletion outcomes, the INDEL profiles are nonetheless heterogeneous in their outcome and are site-specific. NHEJ-based INDEL correction thus may produce both non-productive edits and productive edits in restoring the ORF. Prime editing has an advantage of specifying the exact insertion or deletion outcome for exon reframing, thereby ensuring that all of the edits are productive in restoring the correct ORF. Furthermore, in NHEJ-based INDEL correction, a non-productive edit prevents the sgRNA from re-annealing to the site and inducing a productive edit. In prime editing, a non-productive event (i.e. no editing as the edited strand is not successfully incorporated leaving the native sequence intact) leaves the sgRNA target site still amenable to re-annealing and another attempt at inducing the desired edit.

Prime editing can theoretically be used to correct all possible point mutations including base pair transitions and transversions, whereas base editors are limited only to transitions of A:T to G:C or C:G to T:A. In addition, theoretically prime editing is not limited to an editing window as base editing. Also, prime editing can be used to destroy splice sites. As prime editing necessitates the coordination of multiple pegRNA components for editing, such as the spacer sequence, the primer binding site (PBS), and the reverse transcriptase (RT) template, it is likely that editing events at off-target sites are minimal. However, a recent study demonstrated that two opposite strand nicks using the PE3 system can cause undesired editing outcomes in mouse zygote injections (Aida et al., 2020). These undesired editing outcomes were reduced by utilizing a sgRNA that is mutation-specific and can nick only after successful editing and resolution of the pegRNA nick (PE3b system). Nucleotide editing technologies have the potential to eliminate disease-causing mutations following a single treatment.

PASSIGE (prime-editing-assisted site-specific integrase gene editing) is a prime editing approach, utilizing twinPE, that installs recombinase “landing pads” in the genome and then uses natural recombinase enzymes such as Bxb1 to catalyze the insertion of new DNA into the prime edited target sites.

PASSIGE installs edits in only a small fraction of cells, which is enough to treat some genetic diseases that result from the loss of a functioning gene. In an effort to boost PASSIGE's editing efficiency, the developers found that the recombinase enzyme Bxb1 was a problem in limiting the efficiency of PASSIGE. They then used a tool called PACE (phage-assisted continuous evolution) to rapidly evolve more efficient versions of Bxb1 in the lab.

The resulting engineered Bxb1 variant (eeBxb1) improved the PASSIGE method, thus called eePASSIGE, to integrate an average of 30 percent of gene-sized cargo in mouse and human cells, four times more than the original PASSIGE technique. The newer system provides a better opportunity to efficiently insert large pieces of DNA thousands of base pairs in length at specific sites in the genome. As such, one can make gene-sized edits several times more efficiently than ever before.

TALENs. Transcription activator-like effector nucleases (TALENs) are specific DNA-binding proteins that feature an array of 33 or 34-amino acid repeats. TALENs are artificial restriction enzymes designed by fusing the DNA cutting domain of a nuclease to TALE domains, which can be tailored to specifically recognize a unique DNA sequence. These fusion proteins serve as readily targetable “DNA scissors” for gene editing applications that enable to perform targeted genome modifications such as sequence insertion, deletion, repair and replacement in living cells.

The DNA binding domains, which can be designed to bind any desired DNA sequence, come from TAL effectors, DNA-binding proteins excreted by plant pathogenic Xanthomanos app. TAL effectors consist of repeated domains, each of which contains a highly conserved sequence of 34 amino acids, and recognize a single DNA nucleotide within the target site. The nuclease can create double strand breaks at the target site that can be repaired by error-prone non-homologous end-joining (NHEJ), resulting in gene disruptions through the introduction of small insertions or deletions. Each repeat is conserved, with the exception of the so-called repeat variable di-residues (RVDs) at amino acid positions 12 and 13. The RVDs determine the DNA sequence to which the TALE will bind. This simple one-to-one correspondence between the TALE repeats and the corresponding DNA sequence makes the process of assembling repeat arrays to recognize novel DNA sequences straightforward. These TALEs can be fused to the catalytic domain from a DNA nuclease, FokI, to generate a transcription activator-like effector nuclease (TALEN). The resultant TALEN constructs combine specificity and activity, effectively generating engineered sequence-specific nucleases that bind and cleave DNA sequences only at pre-selected sites. The TALEN target recognition system is based on an easy-to-predict code. TAL nucleases are specific to their target due in part to the length of their 30+ base pairs binding site. TALEN can be performed within a 6 base pairs range of any single nucleotide in the entire genome.

TALEN constructs are used in a similar way to designed zinc finger nucleases (see below) and have three advantages in targeted mutagenesis: (1) DNA binding specificity is higher, (2) off-target effects are lower, and (3) construction of DNA-binding domains is easier.

Zinc Finger Nucleases. As opposed to meganucleases, the concept behind Zinc Finger Nucleases (ZFNs) is based on a non-specific DNA cutting catalytic domain, which can then be linked to specific DNA sequence recognizing peptides such as zinc fingers. The first step to this was to find an endonuclease whose DNA recognition site and cleaving site were separate from each other, a situation that is not the most common among restriction enzymes. Once this enzyme was found, its cleaving portion could be separated which would be very non-specific as it would have no recognition ability. This portion could then be linked to sequence recognizing peptides that could lead to very high specificity.

Zinc finger motifs occur in several transcription factors. The zinc ion, found in 8% of all human proteins, plays an important role in the organization of their three-dimensional structure. In transcription factors, it is most often located at the protein-DNA interaction sites, where it stabilizes the motif. The C-terminal part of each finger is responsible for the specific recognition of the DNA sequence. The recognized sequences are short, made up of around 3 base pairs, but by combining 6 to 8 zinc fingers whose recognition sites have been characterized, it is possible to obtain specific proteins for sequences of around 20 base pairs. It is therefore possible to control the expression of a specific gene. It has been demonstrated that this strategy can be used to promote angiogenesis in animals. It is also possible to fuse a protein constructed in this way with the catalytic domain of an endonuclease in order to induce a targeted DNA break, and therefore to use these proteins as genome engineering tools

The method generally adopted for this involves associating two DNA binding proteins each containing 3 to 6 specifically chosen zinc fingers-with the catalytic domain of the FokI endonuclease which need to dimerize to cleave the double-strand DNA. The two proteins recognize two DNA sequences that are a few nucleotides apart. Linking the two zinc finger proteins to their respective sequences brings the two FokI domains closer together. FokI requires dimerization to have nuclease activity and this means the specificity increases dramatically as each nuclease partner would recognize a unique DNA sequence. To enhance this effect, FokI nucleases have been engineered that can only function as heterodimers.

Several approaches are used to design specific zinc finger nucleases for the chosen sequences. The most widespread involves combining zinc-finger units with known specificities (modular assembly). Various selection techniques, using bacteria, yeast or mammal cells have been developed to identify the combinations that offer the best specificity and the best cell tolerance. Although the direct genome-wide characterization of zinc finger nuclease activity has not been reported, an assay that measures the total number of double-strand DNA breaks in cells found that only one to two such breaks occur above background in cells treated with zinc finger nucleases with a 24 bp composite recognition site and obligate heterodimer FokI nuclease domains.

The heterodimer functioning nucleases would avoid the possibility of unwanted homodimer activity and thus increase specificity of the DSB. Although the nuclease portions of both ZFNs and TALEN constructs have similar properties, the difference between these engineered nucleases is in their DNA recognition peptide. ZFNs rely on Cys2-His2 zinc fingers and TALEN constructs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. Each finger of the Zinc finger domain is completely independent and the binding capacity of one finger is impacted by its neighbor. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

RNA editors. RNA editing has been observed in some tRNA, rRNA, mRNA, or miRNA molecules of eukaryotes and their viruses, archaea, and prokaryotes. RNA editing occurs in the cell nucleus, as well as within mitochondria and plastids. In vertebrates, editing is rare and usually consists of a small number of changes to the sequence of the affected molecules. In other organisms, such as squids, [6] extensive editing (pan-editing) can occur; in some cases the majority of nucleotides in an mRNA sequence may result from editing. More than 160 types of RNA modifications have been described so far.

Unlike DNA editing, which is permanent, the effects of RNA editing—including potential off-target mutations in RNA—are transient and are not inherited. RNA editing is therefore considered to be less risky. Furthermore, it may only require a guide RNA by using the ADAR protein already found in humans and many other eukaryotes' cells instead of needing to introduce a foreign protein into the body.

RNA-editing processes show great molecular diversity, and some appear to be evolutionarily recent acquisitions that arose independently. The diversity of RNA editing phenomena includes nucleobase modifications such as cytidine (C) to uridine (U) and adenosine (A) to inosine (I) deaminations, as well as non-template nucleotide additions and insertions. RNA editing in mRNAs effectively alters the amino acid sequence of the encoded protein so that it differs from that predicted by the genomic DNA sequence.

RNA editing through the addition and deletion of uracil has been found in kinetoplasts from the mitochondria of Trypanosoma brucei. Because this may involve a large fraction of the sites in a gene, it is sometimes called “pan-editing” to distinguish it from topical editing of one or a few sites.

Pan-editing starts with the base-pairing of the unedited primary transcript with a guide RNA (gRNA), which contains complementary sequences to the regions around the insertion/deletion points. The newly formed double-stranded region is then enveloped by an editosome, a large multi-protein complex that catalyzes the editing. The editosome opens the transcript at the first mismatched nucleotide and starts inserting uridines. The inserted uridines will base-pair with the guide RNA, and insertion will continue as long as A or G is present in the guide RNA and will stop when a C or U is encountered. The inserted nucleotides cause a frameshift, and result in a translated protein that differs from its gene.

The mechanism of the editosome involves an endonucleolytic cut at the mismatch point between the guide RNA and the unedited transcript. The next step is catalyzed by one of the enzymes in the complex, a terminal U-transferase, which adds Us from UTP at the 3′ end of the mRNA. The opened ends are held in place by other proteins in the complex. Another enzyme, a U-specific exoribonuclease, removes the unpaired Us. After editing has made mRNA complementary to gRNA, an RNA ligase rejoins the ends of the edited mRNA transcript. As a consequence, the editosome can edit only in a 3′ to 5′ direction along the primary RNA transcript. The complex can act on only a single guide RNA at a time. Therefore, a RNA transcript requiring extensive editing will need more than one guide RNA and editosome complex.

The editing involves cytidine deaminase that deaminates a cytidine base into a uridine base. An example of C-to-U editing is with the apolipoprotein B gene in humans. Apo B100 is expressed in the liver and apo B48 is expressed in the intestines. In the intestines, the mRNA has a CAA sequence edited to be UAA, a stop codon, thus producing the shorter B48 form. C-to-U editing often occurs in the mitochondrial RNA of flowering plants. Different plants have different degrees of C-to-U editing; for example, eight editing events occur in mitochondria of the moss Funaria hygrometrica, whereas over 1,700 editing events occur in the lycophytes Isoetes engelmanii.^{[50]} C-to-U editing is performed by members of the pentatricopeptide repeat (PPR) protein family. Angiosperms have large PPR families, acting as trans-factors for cis-elements lacking a consensus sequence; Arabidopsis has around 450 members in its PPR family. There have been a number of discoveries of PPR proteins in both plastids and mitochondria.

Adenosine-to-inosine (A-to-I) modifications contribute to nearly 90% of all editing events in RNA. The deamination of adenosine is catalyzed by the double-stranded RNA-specific adenosine deaminase (ADAR), which typically acts on pre-mRNAs. The deamination of adenosine to inosine disrupts and destabilizes the dsRNA base pairing, therefore rendering that particular dsRNA less able to produce siRNA, which interferes with the RNAi pathway.

The wobble base pairing causes deaminated RNA to have a unique but different structure, which may be related to the inhibition of the initiation step of RNA translation. Studies have shown that I-RNA (RNA with many repeats of the I-U base pair) recruits methylases that are involved in the formation of heterochromatin and that this chemical modification heavily interferes with miRNA target sites. There is active research into the importance of A-to-I modifications and their purpose in the novel concept of epitranscriptomics, in which modifications are made to RNA that alter their function. A long-established consequence of A-to-I in mRNA is the interpretation of I as a G, therefore leading to functional A-to-G substitution, e.g., in the interpretation of the genetic code by ribosomes. Newer studies, however, have weakened this correlation by showing that inosines can also be decoded by the ribosome (although in a lesser extent) as adenosines or uracils. Furthermore, it was shown that I's lead to the stalling of ribosomes on the I-rich mRNA.

The development of high-throughput sequencing in recent years has allowed for the development of extensive databases for different modifications and edits of RNA. RADAR (Rigorously Annotated Database of A-to-I RNA editing) was developed in 2013 to catalog the vast variety of A-to-I sites and tissue-specific levels present in humans, mice, and flies. The addition of novel sites and overall edits to the database are ongoing. The level of editing for specific editing sites, e.g. in the filamin A transcript, is tissue-specific. The efficiency of mRNA-splicing is a major factor controlling the level of A-to-I RNA editing. Interestingly, ADAR1 and ADAR2 also affect alternative splicing via both A-to-I editing ability and dsRNA binding ability.^{[60][6]}

Alternative U-to-C mRNA editing was first reported in WT1 (Wilms Tumor-1) transcripts, and non-classic G-A mRNA changes were first observed in HNRNPK (heterogeneous nuclear ribonucleoprotein K) transcripts in both malignant and normal colorectal samples. ^{[63]} The latter changes were also later seen alongside non-classic U-to-C alterations in brain cell TPH2 (tryptophan hydroxylase 2) transcripts. Although the reverse amination might be the simplest explanation for U-to-C changes, transamination and transglycosylation mechanisms have been proposed for plant U-to-C editing events in mitochondrial transcripts. A recent study reported novel G-to-A mRNA changes in WT1 transcripts at two hotspots, proposing the APOBEC3A (apolipoprotein B mRNA editing enzyme, catalytic polypeptide 3A) as the enzyme implicated in this class of alternative mRNA editing. It was also shown that alternative mRNA changes were associated with canonical WT1 splicing variants, indicating their functional significance.

Directing edits to correct mutated sequences was first proposed and demonstrated in 1995. This initial work used synthetic RNA antisense oligonucleotides complementary to a pre-mature stop codon mutation in a dystrophin sequence to activate A-to-I editing of the stop codon to a read through codon in a model xenopus cell system. While this also led to nearby inadvertent A-to-I transitions, A to I (read as G) transitions can correct all three stop codons, but cannot create a stop codon. Therefore, the changes led >25% correction of the targeted stop codon with read through to a downstream luciferase reporter sequence. Follow on work by Rosenthal achieved editing of mutated mRNA sequence in mammalian cell culture by directing an oligonucleotide linked to a cytidine deaminase to correct a mutated cystic fibrosis sequence. More recently, CRISPR-Cas 13 fused to deaminases has been employed to direct mRNA editing. In 2022, therapeutic RNA editing for Cas7-11 was reported. It enables sufficiently targeted cuts and an early version of it was used for in vitro editing in 2021.

### II. Detectable Markers

In accordance with the present disclosure, detectable proteins, or proteins that produce a detectable signal, or are otherwise detectable through an interaction with another agent, are employed. Regardless, the detectable agent/event must only occur when the protein is present following a nucleic acid editing event. The signal may be light, color, radioactive emission, proton spin, change in size or mobility through a medium, a chemical change or any other detectable event or condition. This can be performed in situ at the site of disease by inserting genes that encode peptides or proteins that naturally are retained in, on, or near the producing cell, are modified to remain localized (e.g., via a membrane localization sequence or ECM-binding sequence), are naturally secreted for systemic detection, or are engineered to be secreted (e.g., via a signal peptide sequence).

In one aspect, almost all proteins are detectable immunologically, i.e., detectable using an immune receptor, normally an antibody or an antibody fragment. Proteins can be detected when presented on a membrane, in a so-called “membrane bound” form. Alternatively, proteins can be detected when free from membrane involvement. Detection may be purely via the interaction of the immune receptor and protein, but more commonly the immune receptor/antibody is itself labeled with a detectable agent, or even more commonly the primary immune receptor/antibody is itself bound by a “secondary” agent, such as a second immune receptor/antibody, wherein the second immune receptor/antibody is labeled. Such labels include dyes, radiolabels, chemiluminescent agents, fluorophores, etc.

A particularly useful target is bindin, an insoluble protein coating the sperm acrosome process and mediating the adhesion of sperm to sea urchin eggs. Bindin proteins contain a highly conserved central domain flanked by much more highly divergent amino-and carboxyl-terminal domains. It is believed that these conserved domains may participate in membrane fusion and sulfated fucan binding activities. Species-specific adhesion activity appears to be duplicated in both the amino-and carboxyl-terminal domain and may correspond to repeated sequence motifs found in these domains.

Another class of detectable proteins that generate a signal, either on their own or enzymatically through the conversion of a substrate from a non-signal to signal-producing state. Examples include glucose oxidase, urease, creatine deaminase, lipases, alkaline phosphatase, horse radish peroxidase, and beta-galactosidase.

A particular class of enzymes, known as luciferase, are oxidative enzymes that produce bioluminescence from the substrate luciferin, and is usually distinguished from a photoprotein. Luciferases are widely used in biotechnology, for bioluminescence imaging microscopy and as reporter genes, for many of the same applications as fluorescent proteins. However, unlike fluorescent proteins, luciferases do not require an external light source to generate light, but do require addition of luciferin, the consumable substrate.

A variety of organisms regulate their light production using different luciferases in a variety of light-emitting reactions. The majority of studied luciferases have been found in animals, including fireflies, and many marine animals such as copepods, jellyfish, and the sea pansy. However, luciferases have been studied in luminous fungi, like the Jack-O-Lantern mushroom, as well as examples in other kingdoms including bioluminescent bacteria, and dinoflagellates. Engineered forms of luciferases and other enzymes have also been produced, which may be useful in extending their half-life (e.g., via the attachment of an Fc domain of an antibody), increasing their efficiency for better potential sensitivity, reducing their potential immunogenicity, limiting the size of the corresponding gene to enable more efficient insertion, or conferring some other advantageous property.

It may be beneficial to omit a promoter in the donor nucleic acid sequence and instead rely on the upstream promoter of the endogenous gene in which the donor is inserted to minimize the insert for the purposes of insertion efficiency and the elimination of protein production prior to integration. However, the donor gene could also encompass a constitutively active promoter or an inducible promoter to increase protein production and, in the latter case, to control the timing of production. It should also be noted that the initial donor gene inserted need not directly be the detectable gene in this approach. For example, an initial edit of a cell could, instead of directly introducing a donor gene of interest, introduce a recombinase site (e.g., attB, attP) that, in a subsequent step, could be converted to a gene encoding a detectable marker peptide or protein. This approach is especially useful when large genes are desired, which cannot be efficiently inserted via other means.

In some embodiments, the marker peptide or protein is modified to contain a reverse transcytosis domain. Transcytosis is a type of transcellular transport in which various macromolecules are transported across the interior of a cell. Macromolecules are captured in vesicles on one side of the cell, drawn across the cell, and ejected on the other side. Reverse transcytosis is the opposite of this phenomenon, namely, where a specific, receptor-mediated pathway transporting macromolecules from the basolateral (blood/tissue) side to the apical (lumen/mucosa) side. Domains and receptors include Dectin-1 and Siglec-5 for SIgA, TOSO/FAIM3 for IgM, and FcRn for IgG.

### III. Methods of Detection, Diagnosis, Screening and Treatment

The disclosed methods may be applied in a variety of different contexts using variations in tools and timing. Some various applications and a general, non-limiting outline of use cases of this technology are set out below.

Broad screening. In one aspect, one can administer a preset (i.e., non-custom) cocktail of genome-editing formulations each targeting a different oncogenic mutation with donor nucleic acid with the potential for insertion upon cutting. This results in a subset of cancer cells expressing the synthetic biomarker of interest in a secreted format that will accumulate in blood or urine. In the former case, blood will be collected and the synthetic biomarker measured. Enzymes, such as luciferase, are an attractive biomarker for this approach such that simply adding the substrate and reading out bioluminescence in a microplate reader or similar device will deliver the result.

Localization of cancer (e.g., after testing positive during broad screening). In this approach, one will administer a preset (i.e., non-custom) cocktail of genome-editing formulations, each targeting a different oncogenic mutation. This results in a subset of cancer cells expressing the synthetic biomarker of interest in a cell membrane-bound format to remain at the location of the cancer cell expressing it. One can then perform imaging using a contrast agent targeted to the synthetic biomarker. One example of this is the biomarker urease and hyperpolarized MRI due to its exceptional specificity and sensitivity. Others could include antibody-antigen binding or SpyCatcher-SpyTag bonding with a contrast agent on the former component in each pairing.

Cancer staging. This approach differs from the prior ones by starting with the knowledge of the mutation(s) present in a patient's tumor due to, for example, a previous biopsy and sequencing. A custom mutation-targeting formulation for cells harboring one or several mutations is then created that produces a synthetic biomarker on expressed on the cell membrane. This formulation is then administered either intratumorally or intravenously and imaging is performed using either a contrast agent targeted to the synthetic biomarker to determine the extent and location of tumor spreading, choose the right intervention accordingly.

Assessing therapeutic efficacy. Again, as with the staging embodiment, this approach uses a custom mutation-targeting formulation targeting cells harboring one or several mutations based on knowledge of that patient's mutations in order to selectively produce a secreted synthetic biomarker in cells harboring the target mutation(s). This formulation is administered, again either intratumorally or intravenously, and blood is collected at different time points before, during, and after therapeutic intervention to observe the efficacy of the treatment (higher biomarker levels=more cancer cells =less effective therapies).

Detecting Recurrence. Here, the approach begins with having prior knowledge of the mutations present in a patient's cancer, followed by the creation of a custom mutation-targeting formulation for cells harboring one or several mutations that produces either a secreted, cell membrane-bound, or extracellular matrix-bound synthetic biomarker. The formulation is administered either intratumorally or intravenously and the samples (e.g., blood, urine) are examined, optionally with imaging to determine if tumor cells are present. Urine testing may prove to be especially useful to enable facile, continuous monitoring in at-home setting.

### IV. Administration and Formulations

The present disclosure relies, in some aspects, on delivery of genetic editing machinery to cells and organisms. Efficiency of delivery impacts the amount of the detectable biomarker produced. The formulations may be delivered as an “all nucleic acid formulation,” where the editing machinery is generated in the cell/subject, “protein-and-nucleic acid” combination (e.g., ribonucleoproteins), or in another configuration that ultimately leads to provision of the necessary protein and nucleic acid components to cells of interest in a timely and sufficient manner. Depending on the nature of these materials, they may be efficiently delivered in a variety of formulations, including viral vectors or lipid nanoparticles.

As such, the present disclosure provides for pharmaceutical compositions. Such compositions comprise an effective amount of an agent and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the agent, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation. Administration may also be intra-tumoral, into tumor vasculature or region to a tumor site.

Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, ingredients of the compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### V. Kits

In still further embodiments, the present disclosure concerns kits for the labeling of cancer cells in accordance with any of methods described above. The kit may comprise gene editing tools, and may include specific agents that are designed to provide for the labeling of cancer cells with specific mutations, such as gRNAs designed to target such mutations. The kit may also contain constructs for the introduction of biomarkers, i.e., biomarker encoding nucleic acids.

The kits may also include substrates for enzymes that produce detectable signals from said substrates, and/or primary antibodies that recognize and bind antigenic markers, and/or secondary antibodies that are detectably labeled and that bind primary antibodies. The kits may also contain trays, plates, vials, tubes, substrates and membranes for use in detection assays, such as ELISAs, lateral flow assays, and radioassays.

The kits may further comprise suitably aliquoted compositions of any of the foregoing materials, including agents be used to prepare a standard curve for any detection assay. The components of the kits may be packaged either in aqueous media or in lyophilized form. Directions for use in accordance with the disclosed methods may also be included.

The container means of the kits will generally include a means for containing the antibody, antigen, and any other reagent containers in close confinement for commercial sale. Such containers may include injection or blow-molded plastic containers into which the desired vials are retained.

### VI. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

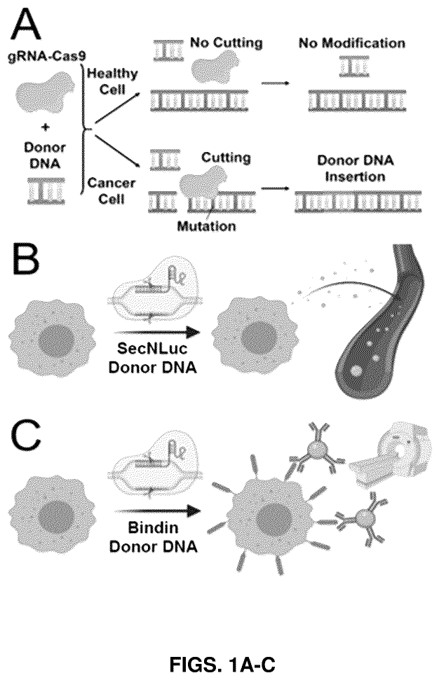

The inventors hypothesized that the single-nucleotide specificity of protospacer adjacent motif (PAM) site recognition by Cas9 variants, combined with the insertion of DNA encoding a unique protein biomarker, could enable the detection of tumors at an early stage, enabling earlier treatment and improved patient outcomes. Briefly, a ribonucleoprotein (RNP) consisting of a Cas9 variant and guide RNA (gRNA) customized to a specific oncogenic mutation can be co-delivered with promoter-less donor DNA encoding an exogenous protein biomarker (FIG. 1A). These components will enter both normal and cancer cells but will only cut the genome in cancer cells that harbor the oncogenic nucleotide sequence defined by the Cas9 variant and gRNA. Because CRISPR is somewhat permissive of single-base mismatches in PAM-distal gRNA and the most common mutations found in cancer are single-base pair substitutions (Ostroverkhova et al., 2023), the preferred approach would be to leverage the high specificity of PAM site recognition using a PAM site that emerges as the result of a mutation and is therefore not present in the wild-type sequence, which is far less permissive to mismatches (Li et al., 2016). In this approach, the mutation itself generates a novel PAM site, enabling the cancer cell genome to be cut while leaving cells with the wild-type sequence unedited. Once cut, donor DNA encoding an exogenous protein will be inserted via homology-directed repair, resulting in the production of the exogenous protein that is ripe for detection. When there are not high-fidelity endonucleases available for PAM-mediated targeting, formulations in which the gRNA has a mismatch with the wild-type sequence in a PAM-proximal position will be prioritized.

The inventors propose in one aspect the use of two genes encoding secreted nanoluciferase (secNLuc), a bioluminescence-producing enzyme (Hall et al., 2012), or bindin, a cell surface protein (Wessel et al., 2021) (FIGS. 1B-C). Both are distinct from human proteins and rather small at 18 kD and 30 kD, respectively, promoting more efficient integration. Since secNLuc is released from the cell, it can be recovered in blood and exposed to its substrate to detect bioluminescence. In contrast, bindin will remain on the cell surface and can be targeted using a bindin antibody-functionalized gadolinium contrast agent and imaged via magnetic resonance imaging (MRI) to identify the location of cancer cells.

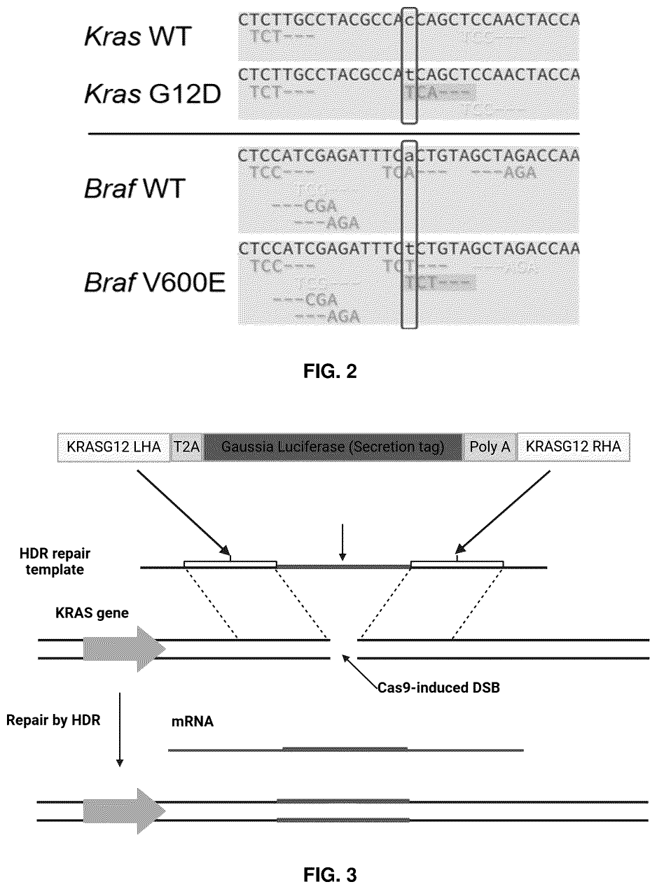

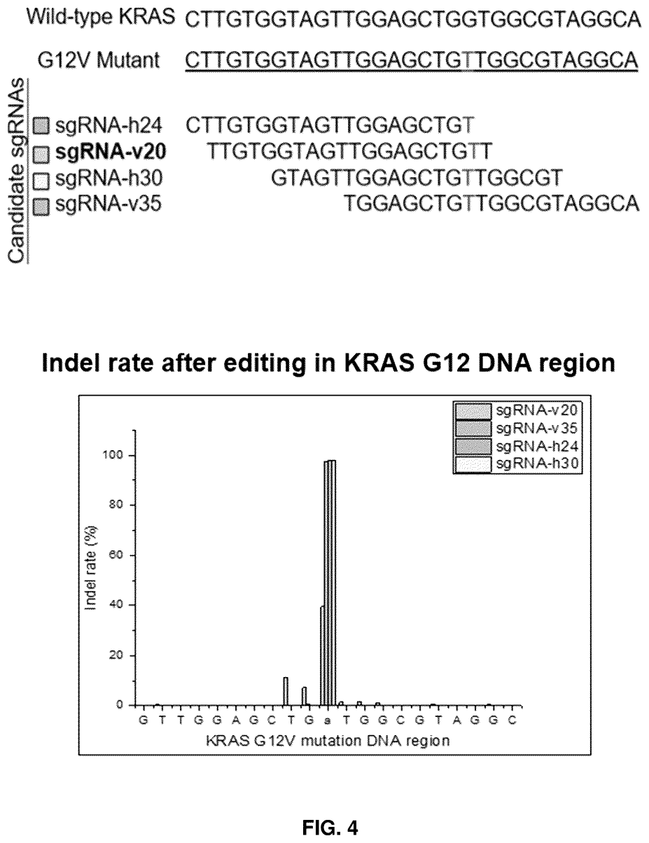

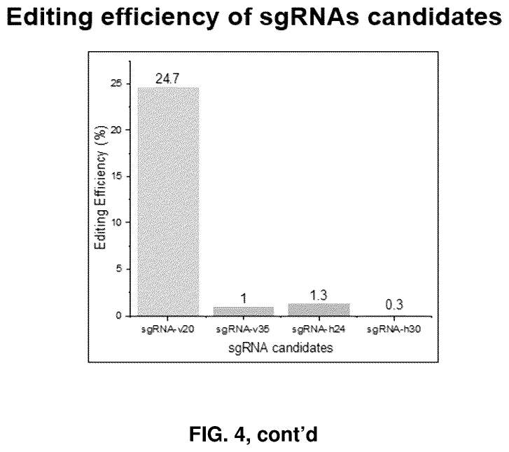

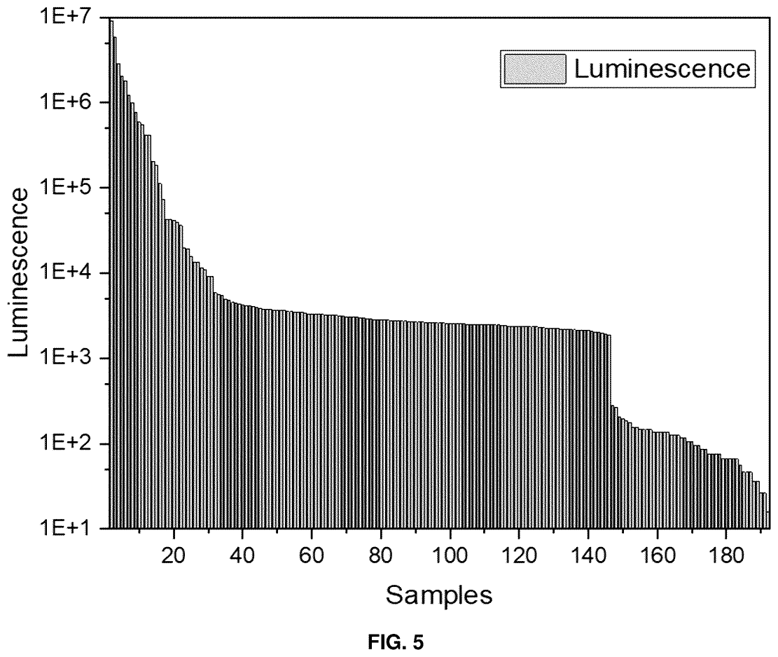

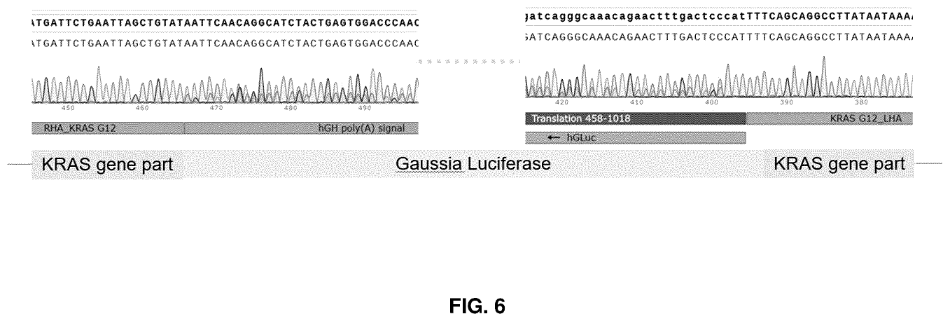

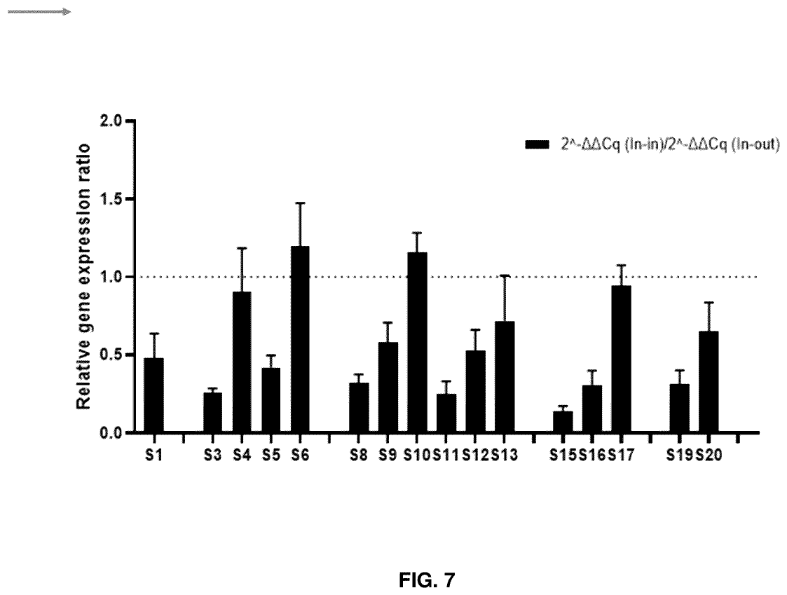

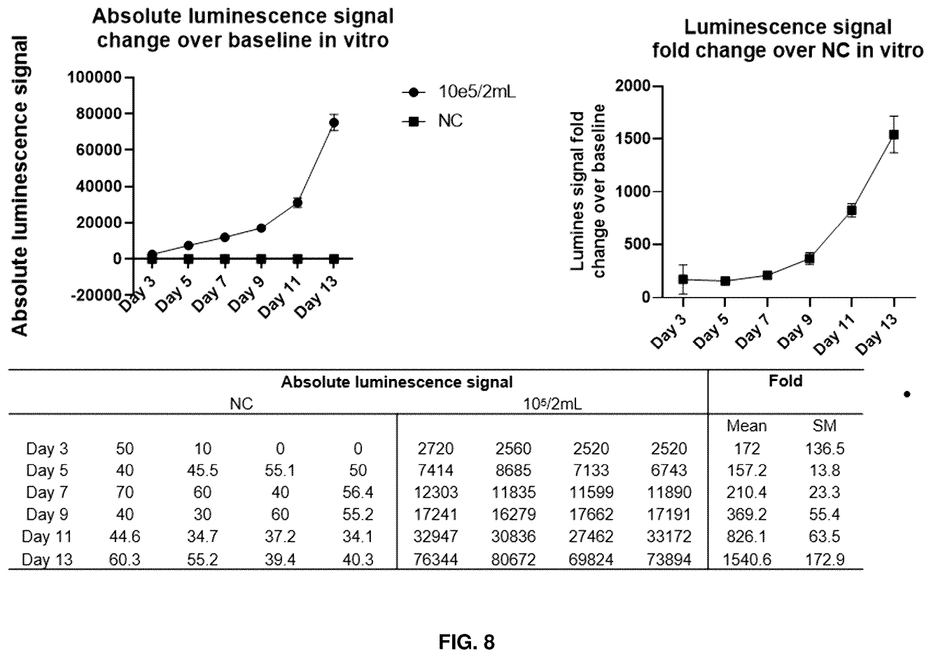

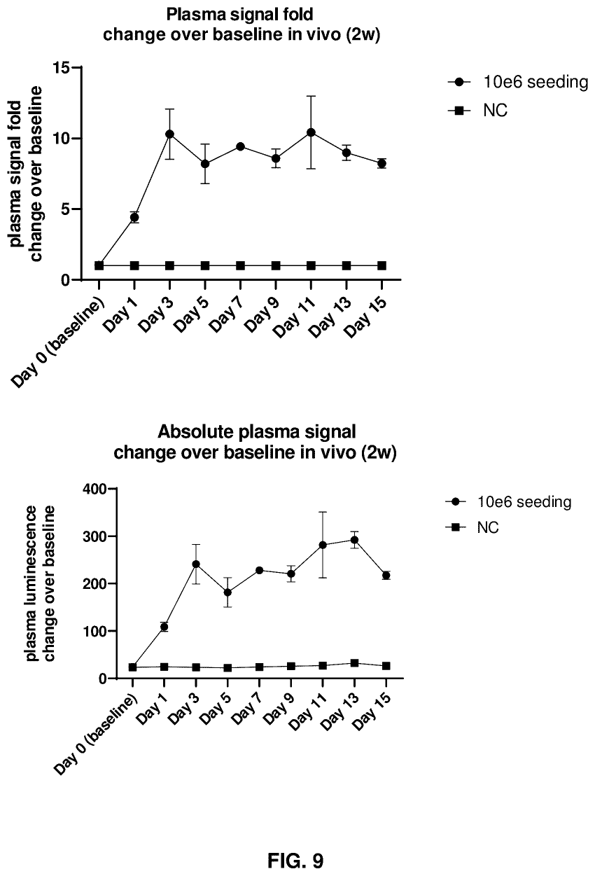

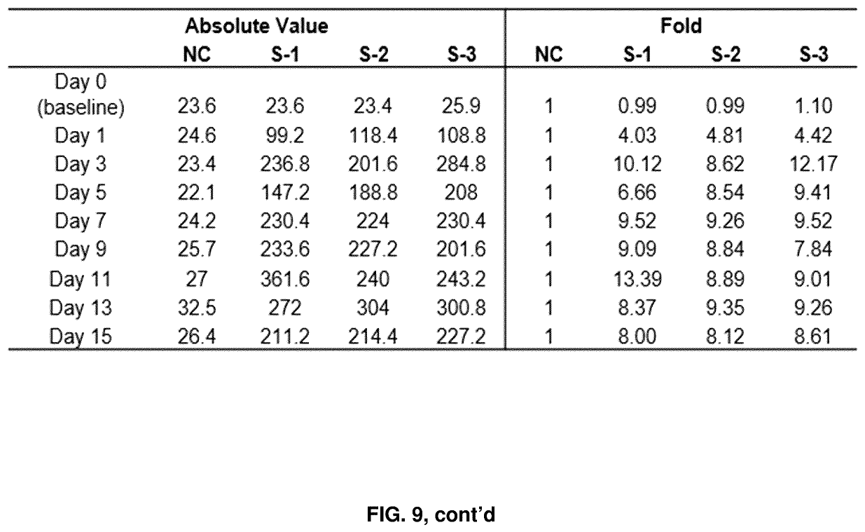

In one approach, the inventors looked at pancreatic cancer, which causes 8% of all cancer deaths despite accounting for only 3% of cancer diagnoses due to frequent late-stage diagnosis and limited treatment options (Li et al., 2016). 33% of all pancreatic cancers feature a single gain-of-function mutation, KRAS G12D, in which a thymine replaces a cytosine, leading to enhanced cell proliferation (Almoguera et al., 1988; Timar & Kashofer, 202). This mutation results in the emergence of a VQR Cas9 PAM site on the reverse complement, which can be exploited to achieve PAM-mediated selective insertion of genes into cancer cells (FIG. 2). In parallel, they will also employ this strategy to target pancreatic cells harboring a very common cancerous mutation, BRAF V600E (NCI Genomic Data Commons), which is an adenine-to-thymine substitution that is the most common cancerous mutation. This mutation similarly leads to the emergence of a VQR Cas9 PAM site that can be exploited to selectively insert a secNLuc or bindin into cells harboring the mutation. Both sites have high (97 and 99 out of 100) cutting frequency determination (CFD) scores, suggesting minimal off-target effects (Doench et al., 2016; Tycko et al., 2019).

The use of CRISPR-Cas9 for differentiating cancer cells from normal cells in vivo based on mutations has ample precedent (Hall et al., 2012; Wessel et al., 2021; Mukhtar et al., 2021; Almoguera et al., 1988; Timar & Kashofer, 2020); however, it has been almost exclusively used for cancer therapy rather than diagnosis. These studies have aimed to either cause an indel in an oncogene (Cheung et al., 2018; Koo et al., 2017; Huang et al., 2018) or, more relevant to this application, inserted a suicide gene from donor DNA (Chen et al., 2017; Martinez-Lage et al., 2020) to kill the cells in vivo. The outstanding results of these studies, including reduced tumor progression and, in one case, an increase in survival from 0% to 100% (Chen et al., 2017), suggest that a diagnostic will be similarly specific and successful. Importantly, in these studies, researchers did not identify harmful off-target editing, though the duration of those studies were limited.

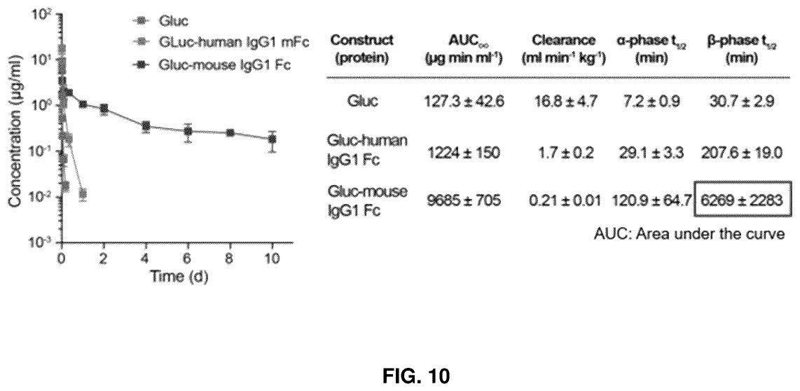

To improve the sensitivity of the cancer detection method, a reverse transcytosis domain was incorporated into the reporter protein, with the aim of extending its half-life and lowering the detection limit (FIG. 10). To evaluate the detection limit of the modified reporter construct, a single-cell clone stably expressing the reporter gene was generated using CRISPR-based genome editing. To verify correct insertion of the reporter gene at the target locus (KRAS G12), two pairs of primers were designed for ddPCR analysis. For the first pair, one primer anneals to the genomic KRAS locus and the other to the reporter gene, enabling detection of site-specific integration. For the second pair, both primers target sequences within the reporter gene to assess its overall expression. Probes were designed within the amplified regions for both primer sets. Gene expression levels were then quantified in candidate clones, and clones exhibiting an expression ratio close to 1 were selected for subsequent experiments.

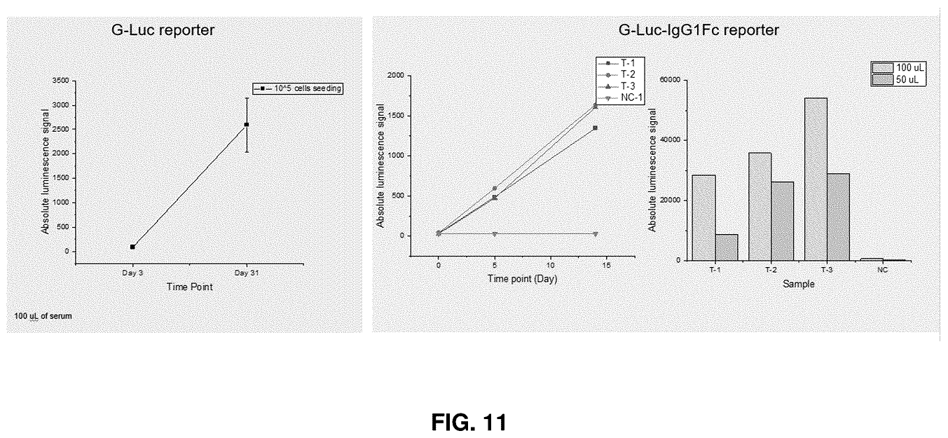

The selected cell clone was subsequently used for in vivo studies. A total of 1×10⁵ cells were subcutaneous injected into mice, and serum samples were collected at multiple time points to evaluate the expression of the modified reporter protein. The results demonstrated that, by Day 35, the engineered reporter construct produced over 20-fold higher signal compared to the reporter lacking the IgG1 Fc domain (FIG. 11). These findings suggested that incorporation of the IgG1 Fc domain significantly enhanced in vivo sensitivity of detection and lower the detection limit.

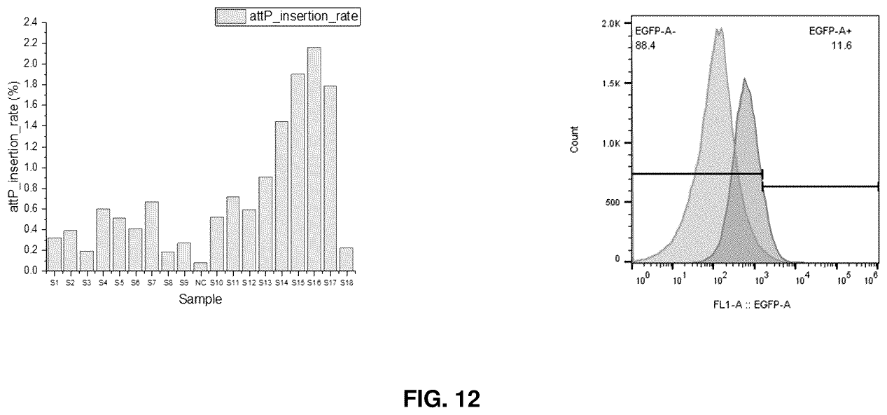

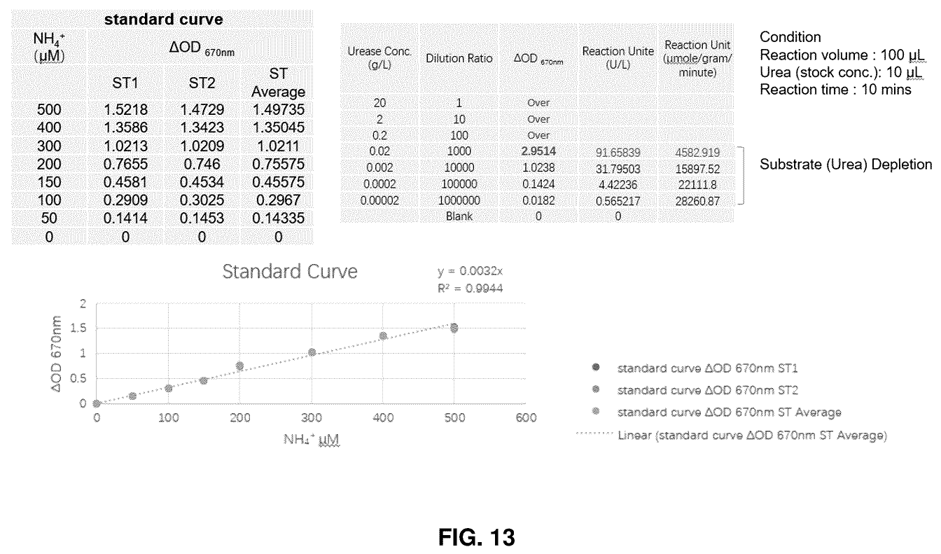

To enhance specificity and in vivo editing efficiency, the inventors explored a novel prime editing-based strategy, termed eePASSIGE, developed by David Liu's lab. Custom twin pegRNAs were designed to mediate insertion of an attP sequence at the KRAS G12 locus in NCI-H727 cells. Indel frequencies were consistently below 0.5% across all pegRNA pairs. The optimal twin pegRNA pair, exhibiting the highest attP insertion efficiency, was selected for subsequent experiments. This optimized pegRNA pair was then combined with Bxb1 recombinase and an attB donor plasmid encoding eGFP. Approximately 11.5% of cells exhibited eGFP expression (FIG. 12), indicating efficient site-specific integration and supporting the potential of this approach as a precise and efficient genome editing strategy. In parallel, we evaluated an alternative reporter system based on urea-urease chemistry for potential detection of small tumor lesions using hyperpolarized MRI. Urease was introduced into cancer cells, and its enzymatic activity was assessed in vitro using a colorimetric assay (FIG. 13).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
GBD 2016 Causes of Death Collaborators. Global, regional, and national age-sex specific mortality for 264 causes of death, 1980-2016: a systematic analysis for the Global Burden of Disease Study 2016. Lancet 390, 1151-1210 (2017).
Crosby, D. et al. Early detection of cancer. Science 375, eaay9040 (2022).
Li, Y. et al. Exploiting the CRISPR/Cas9 PAM Constraint for Single-Nucleotide Resolution Interventions. PLOS One 11, e0144970 (2016).
Home|NCI Genomic Data Commons./gdc.cancer.gov/.
Partecke, L. I. et al. A syngeneic orthotopic murine model of pancreatic adenocarcinoma in the C57/BL6 mouse using the Panc 02 and 6606PDA cell lines. Eur Surg Res 47, 98-107 (2011).
de Souza, J. A., Hunt, B., Asirwa, F. C., Adebamowo, C. & Lopes, G. Global Health Equity: Cancer Care Outcome Disparities in High-, Middle-, and Low-Income Countries. J Clin Oncol 34, 6-13 (2016).
Kaiser, J. ‘Liquid biopsy’ for cancer promises early detection. Science 359, 259 (2018).
Cleeland, C. S. et al. Reducing the toxicity of cancer therapy: recognizing needs, taking action. Nat Rev Clin Oncol 9, 471-478 (2012).
Hazelton, W. D. & Luebeck, E. G. Biomarker-based early cancer detection: is it achievable? Sci Transl Med 3, 109fs9 (2011).
Cohen, J. D. et al. Detection and localization of surgically resectable cancers with a multi-analyte blood test. Science 359, 926-930 (2018).
Ostroverkhova, D., Przytycka, T. M. & Panchenko, A. R. Cancer driver mutations: predictions and reality. Trends Mol Med S1471-4914(23)00067-9 (2023) doi: 10.1016/j. molmed.2023.03.007.
Hall, M. P. et al. Engineered luciferase reporter from a deep sea shrimp utilizing a novel imidazopyrazinone substrate. ACS Chem Biol 7, 1848-1857 (2012).
Wessel, G. M., Wada, Y., Yajima, M. & Kiyomoto, M. Bindin is essential for fertilization in the sea urchin. Proc Natl Acad Sci U S A 118, e2109636118 (2021).
Mukhtar, R. A. et al. Accuracy of sentinel lymph node biopsy in invasive lobular carcinoma of the breast: Factors associated with false negatives. Breast J 27, 406-408 (2021).
Almoguera, C. et al. Most human carcinomas of the exocrine pancreas contain mutant c-K-ras genes. Cell 53, 549-554 (1988).
Timar, J. & Kashofer, K. Molecular epidemiology and diagnostics of KRAS mutations in human cancer. Cancer Metastasis Rev 39, 1029-1038 (2020).
Doench, J. G. et al. Optimized sgRNA design to maximize activity and minimize off-target effects of CRISPR-Cas9. Nat Biotechnol 34, 184-191 (2016).
Tycko, J. et al. Mitigation of off-target toxicity in CRISPR-Cas9 screens for essential non-coding elements. Nat Commun 10, 4063 (2019).
Cheung, A. H.-K. et al. Specific targeting of point mutations in EGFR L858R-positive lung cancer by CRISPR/Cas 9. Lab. Invest. 98, 968-976 (2018).
Koo, T. et al. Selective disruption of an oncogenic mutant allele by CRISPR/Cas9 induces efficient tumor regression. Nucleic Acids Res 45, 7897-7908 (2017).
Huang, L. -C. et al. CRISPR/Cas9 Genome Editing of Epidermal Growth Factor Receptor Sufficiently Abolished Oncogenicity in Anaplastic Thyroid Cancer. Dis Markers 2018, 3835783 (2018).
Chen, Z. -H. et al. Targeting genomic rearrangements in tumor cells through Cas9-mediated insertion of a suicide gene. Nat. Biotechnol. 35, 543-550 (2017).
Martinez-Lage, M. et al. In vivo CRISPR/Cas9 targeting of fusion oncogenes for selective elimination of cancer cells.Nat Commun 11, 5060 (2020).

1. A method of labeling a cancer cell comprising subjecting the cancer cell to gene editing that introduces a genetic element encoding a detectable marker peptide or protein when one or more cancer-specific mutations are present.

2. The method of claim 1, wherein gene editing comprises Cre-Lox recombination, Prime Editing, Base Editing, TALEN editing, zinc finger nuclease editing, CRISPR/Cas editing, e.g., PASSIGE™, eePASSIGE™ or an RNA-editing technique, e.g., Cas13a.

3. The method of claim 1, wherein the detectable marker peptide or protein is a surface-bound or extracellular matrix-bound target, such as bindin, and detecting comprises contacting said cancer cell with an target binding agent, such as antibody, an aptamer, or bioconjugate pair (e.g., SpyTag/SpyCatcher), optionally wherein said target binding agent is labeled with a detectable moiety, or wherein the detectable marker peptide or protein is an intracellular protein.

4. The method of claim 1, wherein the detectable marker peptide or protein is an enzyme, such as beta-lactamase or luciferase.

5. The method of claim 1, wherein the detectable peptide or protein is linked to a reverse-transcytosis domain, e.g., FcRn, Dectin-1, Siglec-5, or TOSO/FAIM3.

6. The method of claim 1, further comprising detecting a signal produced directly or indirectly from the presence of said detectable peptide or protein marker.

7. The method of claim 1, wherein said labeling is performed in vitro on a cancer cell obtained from a subject, optionally wherein detecting comprises an ELISA, lateral flow assay, a bioluminescence assay, chemical assay (e.g., liquid chromatography-mass spectrometry), or absorbance-based assay.

8. The method of claim 1, wherein said labeling is performed in vivo in a subject, such as intratumorally, interperitoneally, or intravenously.

9. The method of claim 8, wherein detecting comprises obtaining a body fluid from the subject, such as blood, saliva, sputum, urine, semen or other bodily secretion, optionally wherein detecting comprises an ELISA, lateral flow assay, a bioluminescence assay, chemical assay (e.g., liquid chromatography-mass spectrometry), or absorbance-based assay, or wherein detecting comprises obtaining an image from the subject.

10. The method of claim 1, wherein the gene editing inserts more than one genetic element encoding more than one detectable marker peptide or protein at more than one mutation site, such as wherein said multiple detectable marker peptides or proteins each produce distinguishable signals.

11. The method of claim 1, wherein a cancer cell from said subject has been sequenced to determine the presence of a mutation.

12. The method of claim 1, further comprising making a diagnosis, treatment decision, or treatment based on a location and/or extent of said signal.

13. The method of claim 12, further comprising performing detecting said signal a second time determining a change in a previously obtained signal following treatment.

14. The method of claim 1, wherein the coding region for said detectable marker peptide or protein is operatively linked to an inducible promoter, a constitutively active promoter, or does not have a promoter operatively linked, or may be induced by triggering of Cre-Lox recombination events.

15. The method of claim 6, wherein said subject was previously diagnosed and treated for cancer, such as wherein said subject has been in remission.

16. The method of claim 6, wherein said subject has not been diagnosed with cancer and has no known cancer risk factors.

17. The method of claim 6, wherein said subject has not been diagnosed with cancer and has one or more known cancer risk factors.

18. The method of claim 6, wherein said cancer cell is contacted with a candidate substance and cultured, wherein a lower signal, as compared to an untreated cancer cell having the same genetic abnormality and cultured under the same conditions, indicates that said candidate substance has anti-cancer activity.

19. The method of claim 9, wherein the target binding agent is labeled with a contrast agent, a radioisotope, a molecular imaging agent, or a nanoparticle.

20. The method of claim 9, wherein imaging comprises detecting an enzymatic signal, a bioluminescent signal, a chemical signal, or an absorbance-based signal.
